(19) United States
(12) Patent Application Publication
(10) Pub. No.: US 2015/0174328 A1
(43) Pub. Date: Jun. 25, 2015

(54) Medicament Container Retaining Mechanism

(71) Applicant: Carebay Europe Ltd, Swatar (MT)

(72) Inventors: Stefan Lööf, Skondal (SE); Niclas Yman, Ekero (SE)

(73) Assignee: Carebay Europe Ltd, Swatar (MT)

(21) Appl. No.: 14/404,537

(22) PCT Filed: May 22, 2013
(86) PCT No.: PCT/EP2013/060444

§ 371 Date: Nov. 28, 2014

Related U.S. Application Data
(60) Provisional application No. 61/652,962, filed on May 30, 2012.

(30) Foreign Application Priority Data
May 29, 2012 (SE) 1250551-7

(51) Int. Cl. A61M 5/24

(52) U.S. Cl. CPC A61M 5/24; A61M 2005/2492

(57) ABSTRACT

A medicament container retaining mechanism for a medicament delivery device includes at least one flexible retaining member arranged to a first housing part of the medicament delivery device. The first housing part is configured to receive a generally tubular medicament container. The at least one flexible retaining member is configured to interact with a rim of the received medicament container, and the at least one flexible retaining member is configured to exert a mainly longitudinally directed clamping force on the rim of the medicament container to prevent movement in the generally longitudinal direction of the medicament container in the housing part.

### TECHNICAL AREA

The present invention relates to a medicament container retaining mechanism for a medicament delivery device, especially an injection device, to prevent rattling of a medicament container or ampoule containing a specific medicament therein.

### BACKGROUND OF INVENTION

Medicament delivery devices that can be manually loaded with a medicament container or assembled to house a pre-specified cartridge therein are known. Conventional injectors built to accommodate a medicament container therein typically include mechanical structures designed to retain or hold the container within the housing of the injectors. Furthermore, a standard cartridge or ampoule containing a specific medicament therein is known to comprise at least the parts of: (a) an outlet end portion with a septum, (b) a barrel portion for containing the medicament and (c) a neck portion connecting the outlet portion and the barrel portion of the cartridge.

To provide proper injections and to withstand the driving force of a driving mechanism of an auto or semi-auto injector, the container received within the housing of the injector needs to be retained firmly or secured properly such that the force-actuated plunger thereof can be driven, thereby driving a stopper of the medicament container accurately towards the proximal end of the injector such that a correct amount of medicament can be delivered. It is known that by providing a mechanical structure to grip on the neck portion of the cartridge, the cartridge can be secured within the housing of the injector and restricting at least the longitudinal or vertical movement of the medicament container. However, such retaining of the medicament container within the housing of the injector may be sufficient to a relatively short ampoule or a cartridge that is short in length. There is a need to prevent undesirable movement or non-securement of the cartridge received in the injector, in particular for cartridges of a greater length, such that undesirable movement in the lateral direction, causing rattling of the container in the injector, can be advantageously prevented for proper injections. Further, when a device is to be made ready for use, a medicament container is placed in a first housing part, which housing part then is connected to a second housing part. There is a pronounced risk that the medicament container will fall out from the first housing part if it is accidentally turned before connection. This problem also needs to be remedied.

### BRIEF DESCRIPTION OF INVENTION

The aim of the present invention is to remedy any drawbacks of medicament delivery devices of the state of the art, and to provide an improved medicament container retaining mechanism.

In the present application, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site. Further, the term “longitudinal”, with or without “axis”, refers to a direction or an axis through the device or components thereof in the direction of the longest extension of the device or the component. In a similar manner, the term “lateral”, with or without “axis”, refers to a direction or an axis through the device or components thereof in a direction generally perpendicular to the longitudinal direction. Also, if nothing else is stated, in the following description wherein the mechanical structure of the device and the mechanical interconnection of its components are described, the device is in an initial non-activated or non-operated state.

The main aspects of the invention are characterised by the features of the independent patent claim.

Preferable embodiments of the invention form the subject of the dependent patent claims.

According to the present invention, there is provided a medicament container retaining mechanism for a medicament delivery device. Preferably it comprises at least one flexible retaining member arranged to a first housing part of said medicament delivery device. The first housing part is configured or designed to receive a generally tubular medicament container therein. The medicament container may be of several types such as syringes, ampules, cartridges, just to mention a few.

According to a preferable aspect of the invention, the at least one flexible retaining member is configured to interact with a rim of said medicament container received therein. The at least one flexible retaining member is configured to exert mainly longitudinally directed clamping forces to said medicament container to prevent movement in the generally longitudinal direction of said medicament container in said housing part.

In this manner, the movement of the medicament container is prevented or at least greatly reduced, whereby any rattling or other noises coming from the movement of the medicament container inside the housing is effectively removed or reduced. Further, the risk of the medicament container falling out of the first housing part is minimized. The device is thereby perceived as being stable and solid by a user, giving a positive impression of quality, and the risk of breaking a medicament container and thereby loosing medicament is clearly reduced.

In addition, said at least one flexible retaining member is configured to exert also laterally directed clamping forces to said medicament container to prevent movement in the generally lateral direction of said medicament container in said housing part. Thus, the flexible retaining member is then capable of effectively preventing any movement of the medicament container within the first housing part.

According to a further preferable solution of the present invention, said at least one flexible retaining member may be integrally formed with said first housing part as one unitary part. This solution facilitates the manufacture of the present invention.

In that aspect, said at least one flexible retaining member may be formed by a generally U-shaped cut-out portion in said housing part, whereby a tongue-like member is obtained. This design also facilitates the manufacturing of the present invention in that the flexible member may be formed as a resilient tongue by the U-shaped cut-out.

Further, preferably the at least one flexible retaining member is arranged with an inwardly directed protrusion, wherein said at least one retaining member is capable of flexing laterally in a direction substantially perpendicular to a longitudinal axis of the housing part such that said protrusion is moved into contact with said rim of the medicament container when inserting the container into the first housing from a distal direction. The protrusion of the retaining member is then capable of providing a stop of any movement in the longitudinal direction since it is in contact with the rim, but preferably also providing a stop for any movement in the lateral direction.

According to a further preferable solution of the present invention, said at least one flexible retaining member may be arranged with an outwardly extending ledge, which ledge is arranged to come in contact with an inner surface of a second housing part, to which the first housing part is attachable, when the housing parts are attached, such that said at least one flexible retaining member is forced radially inwards. With this solution, a positive contact is ascertained between the retaining member and the rim of the medicament container even with tolerance differences regarding the length of the medicament container.

These and other aspects of, and advantages with, the present invention will become apparent from the following detailed description of the invention and from the accompanying drawings.

### BRIEF DESCRIPTION OF DRAWINGS

In the following detailed description of the invention, reference will be made to the accompanying drawings, of which

FIG. 1 is a perspective side-view of a medicament delivery device comprising the present invention,

FIG. 2 is a cross-sectional view of a proximal part of the medicament delivery device of FIG. 1, and

FIGS. 3-5 are detailed views of a housing part arranged with a medicament container retaining mechanism according to the present invention.

### DETAILED DESCRIPTION OF THE INVENTION

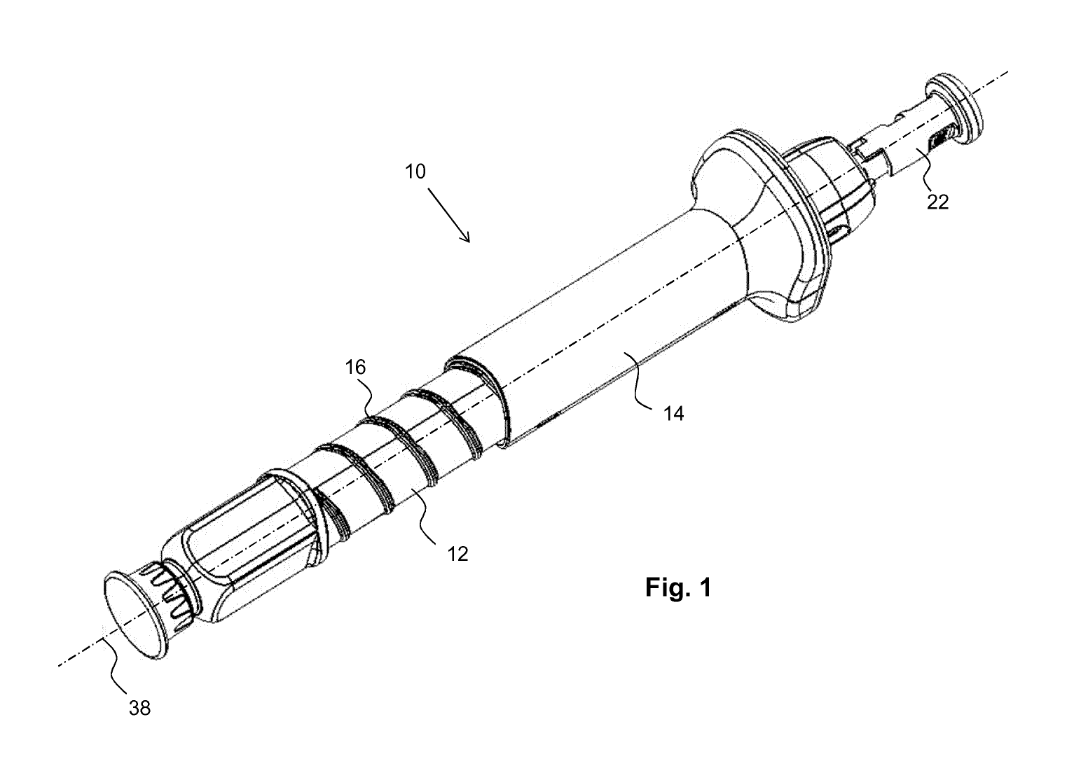

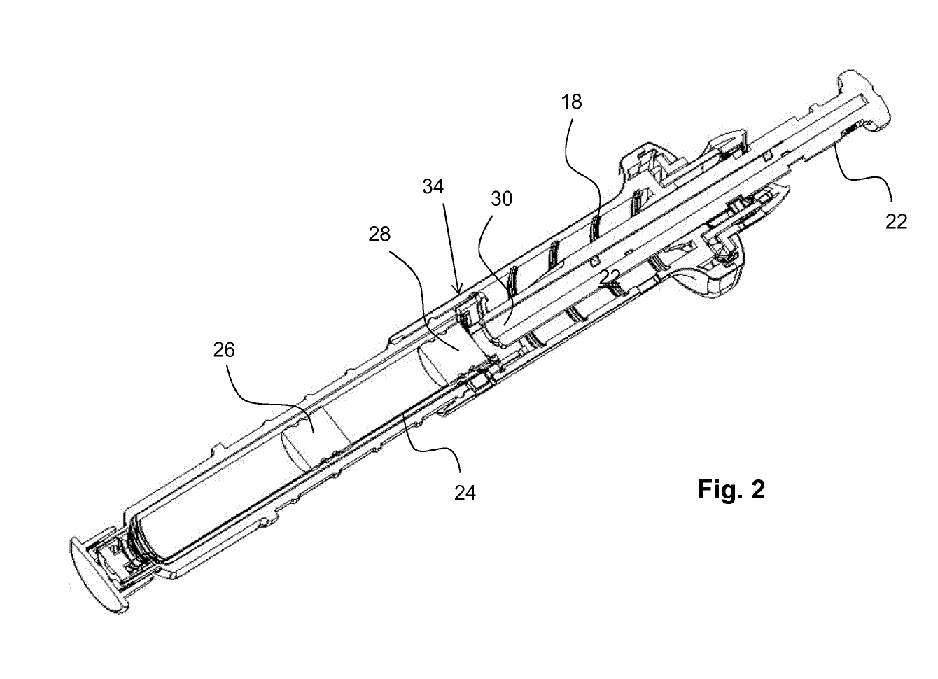

In an exemplary embodiment of a medicament delivery device comprising the present invention, as shown in FIG. 1, the medicament delivery device 10 comprises a first housing part 12 having a generally tubular shape, and a second housing part 14. The first housing part 12 is arranged with attachment means arranged to cooperate with corresponding attachment means on the second housing part 14. As a non-limiting example, in the embodiment shown the attachment means on the first housing part 12 comprises a spirally extending ridge 16 arranged on its outer surface. The ridge 16 cooperates with a mating spirally extending groove 18, FIG. 2, on an inner surface of the second housing part 14, such that the two housing parts may be screwed together. It is however to be understood that a number of other types of attachment means are available to the person skilled in the art, such as bayonet connections, snap-in members etc.

The second housing part 14 further comprises a drive mechanism 20, which drive mechanism may have a manually operated activation member 22 at a distal end of the second housing part 14. Further, the first housing part 12 is designed to accommodate a medicament container 24 containing a specific medicament therein, FIG. 2.

The medicament container 24 comprises at least the parts of: (a) an outlet end portion with a septum or an outlet with a delivery member as e.g. a needle, (b) a barrel portion for containing the medicament, (c) a neck portion connecting the outlet portion and the barrel portion of the cartridge, (d) at least one stopper, and (e) a rim at the distal end of the barrel portion.

As a non-limiting example, the medicament container 24 may be a so-called dual chamber container arranged with two compartments, one with the medicament in powder form and the other with a diluent. The compartments are initially closed by a first generally resilient stopper 26. At a distal end of the medicament container a second stopper 28 is arranged. It is however to be understood that the type of medicament container is not vital for the present invention. The medicament container may as well comprise a single chamber medicament container where the medicament is prepared and ready for delivery.

The drive mechanism is arranged such that manual operation of the activation member 22 causes a plunger rod 30, in the embodiment shown integrated with the activation member, to be moved in the proximal direction of the device, thereby acting on the stopper 28 of the medicament container 24 for either a mixing operation if the medicament container is of dual chamber type, or delivery of a certain dose of medicament from the container.

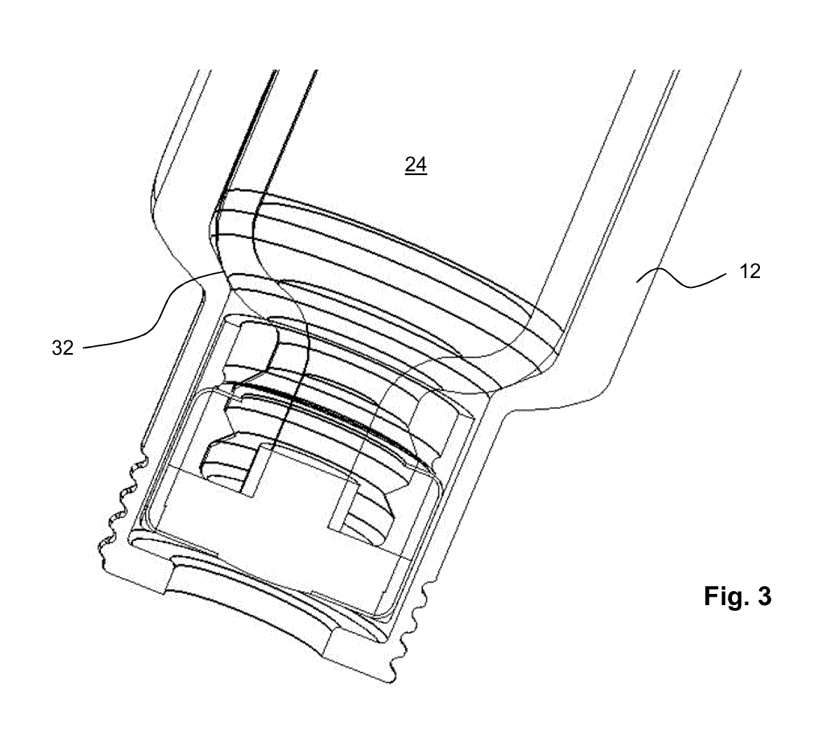

A proximal end surface 32, FIG. 3, of the first housing part 12 is preferably arranged to be in contact with a neck portion of the medicament container 24, providing a stop or reference surface for the medicament container in the proximal direction of the device.

According to the present invention, it comprises a medicament container retaining mechanism 34, FIG. 2. The medicament container retaining mechanism 34 is arranged on the first housing part 12 to facilitate securement of a medicament container 24 entered into the first housing part from a distal direction, and to prevent or avoid movement in a generally longitudinal direction, together with the end surface 32, as well as minimizing movement in the generally lateral direction, preventing any movement such as rattling of the medicament container 24 in the first housing part 12 but also minimizing the risk of the medicament container falling out of the first housing part, if the latter is turned with the distal end facing downwards.

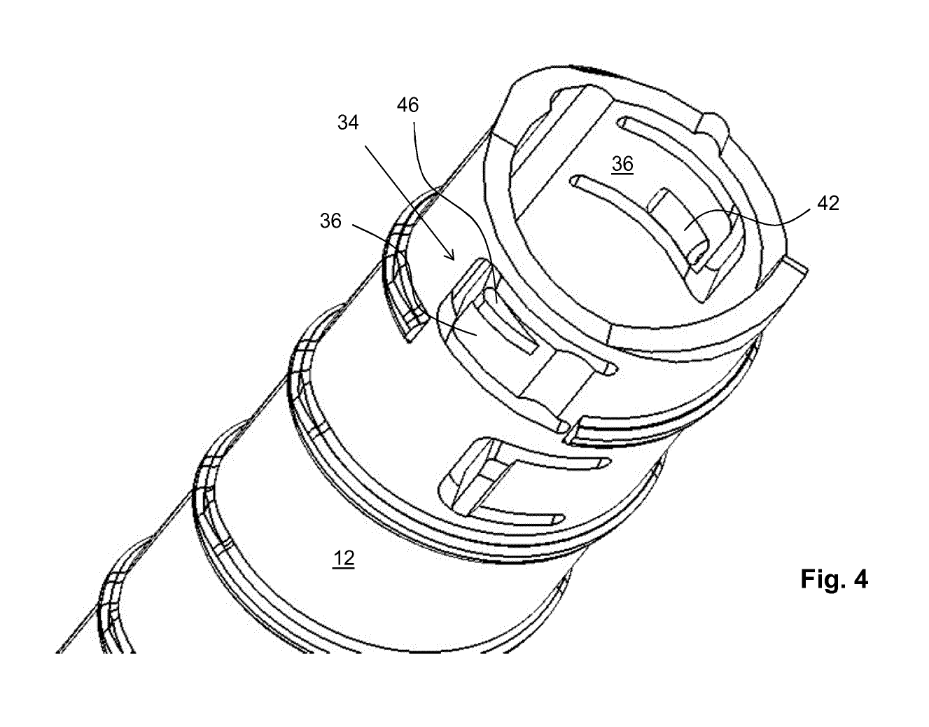

As shown in FIG. 4, the medicament container retaining mechanism 34 is provided on at least a distal portion of the first housing part 12. In a preferred embodiment shown, the first housing part 12 comprises at least one flexible retaining member 36, FIG. 4, preferably two positioned on opposite sides as seen along a longitudinal axis 38, configured to interact or detachably engage with a rim 44 of the generally tubular medicament container 24.

The retaining members 36 are in the embodiment shown formed integral with the material of the first housing part 12 where each retaining member 36 is formed by a generally U-shaped cut-out, FIG. 4, whereby a tongue is formed, extending in the generally circumferential direction of the first housing part 12. In the embodiment shown each tongue is arranged with an inwardly extending protrusion 42. The position of the tongue 36 and the protrusion 42 are chosen in relation to the end surface 32 such that, when a container 24 is placed in the first housing part 12, the rim 44 of the container 24 is in contact with, or in very close proximity to, the protrusions 42 of the tongues, FIG. 4.

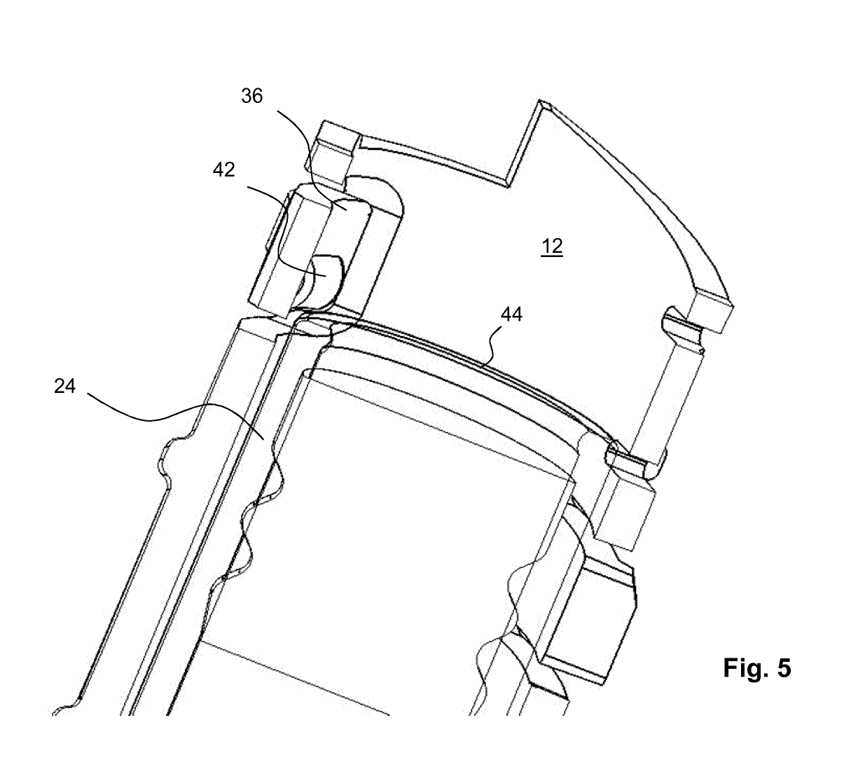

Further, the tongues 36 are arranged with outwardly extending ledges 46, which ledges 46 have a general wedge-shape as seen in a plane transversal to the longitudinal axis 38. The ledges 46 will come in contact with, and slide along, the inner surface of the second housing part 14 when the two housing parts are brought together after the first housing part 12 has been provided with a medicament container 24, where the sliding is facilitated by the wedge-shape. Thus the contact of the ledges 46 on the inner surface will force the tongues 36 generally radially inwards, which in turn will force the protrusions 42 to press on the rim 44 of the medicament container. Alternatively, since the length tolerances of the medicament container 24 may differ somewhat, the inward movement of the protrusions will ensure a contact with the rim 44 of the medicament container. In this respect the protrusions 42 may have different shapes as seen in a cross-sectional view. In the embodiment shown the cross-sections of the protrusions have the shape of half a circle, FIG. 5, but they could also have an inclined contact surface or in other ways varying extension that provides a good contact with the medicament container when the tongues are pressed inwards.

The first housing part 12 including the retaining member 36 may be made of flexible materials such as plastics, metal alloys or combinations thereof, whereby the retaining member 36 of the medicament container retaining mechanism 34 exhibits a resilient property in order to be able to flex in a direction substantially perpendicular to the longitudinal axis 38 of the first housing part 12. It is to be understood that the number of retaining members 36 of the medicament container retaining mechanism 34 is not limited to any number, and there may be provided with more than one pair of retaining members 36 or an odd number of said members 36 on the first housing part 12 of the medicament delivery device 10.

It is to be understood that the embodiment described above and shown in the drawings is to be regarded only as a non-limiting example of the invention and that it may be modified in many ways within the scope of the patent claims.

1-7. (canceled)

8. A medicament container retaining mechanism for a medicament delivery device, comprising:
at least one flexible retaining member arranged to a first housing part of the medicament delivery device, the first housing part being configured to receive a generally tubular medicament container therein;
wherein the at least one flexible retaining member interacts with a rim of a medicament container received in the first housing part, exerting a longitudinally directed clamping force on the rim to prevent movement in a longitudinal direction of the medicament container in the first housing part; the at least one flexible retaining member and the first housing part are a unitary part; and the at least one flexible retaining member is a generally U-shaped cut-out portion in the first housing part having a resilient tongue.

9. The mechanism of claim 8, wherein the at least one flexible retaining member includes an outwardly extending ledge that is configured to contact an inner surface of a second housing part when the first housing part is attached to the second housing part, and when the first and second housing parts are attached, the at least one flexible retaining member is forced radially inward.

10. The mechanism of claim 8, wherein the at least one flexible retaining member exerts a laterally directed clamping force on the medicament container received in the first housing part to prevent movement in a generally lateral direction of the medicament container in the first housing part.

11. The mechanism of claim 10, wherein the at least one flexible retaining member includes an outwardly extending ledge that is configured to contact an inner surface of a second housing part when the first housing part is attached to the second housing part, and when the first and second housing parts are attached, the at least one flexible retaining member is forced radially inward.

12. The mechanism of claim 8, wherein the resilient tongue comprises a radially inwardly directed protrusion, such that when the generally tubular medicament container is received in the first housing part, the protrusion is in contact with the rim of the medicament container.

13. The mechanism of claim 12, wherein the at least one flexible retaining member includes an outwardly extending ledge that is configured to contact an inner surface of a second housing part when the first housing part is attached to the second housing part, and when the first and second housing parts are attached, the at least one flexible retaining member is forced radially inward.

14. The mechanism of claim 8, wherein the mechanism is included in a medicament delivery device.

15. The mechanism of claim 14, wherein the medicament delivery device is an injection device.

16. The mechanism of claim 15, wherein the medicament container is a syringe or a cartridge.

17. The mechanism of claim 10, wherein the mechanism is included in a medicament delivery device.

18. The mechanism of claim 17, wherein the medicament delivery device is an injection device.

19. The mechanism of claim 18, wherein the medicament container is a syringe or a cartridge.

20. The mechanism of claim 12, wherein the mechanism is included in a medicament delivery device.

21. The mechanism of claim 20, wherein the medicament delivery device is an injection device.

22. The mechanism of claim 21, wherein the medicament container is a syringe or a cartridge.
